(19) United States
(12) Patent Application Publication
(10) Pub. No.: US 2015/0141868 A1
(43) Pub. Date: May 21, 2015

(54) NEEDLE BIOPSY SYSTEMS AND METHODS

(71) Applicants: Boston Scientific Scimed, Inc., Maple Grove, MN (US); Mayo Foundation for Medical Education and Research, Rochester, MN (US)

(72) Inventors: Kimberly Clark, Coon Rapids, MN (US); Bruce Forsyth, Hanover, MN (US); Gregory J. Sherwood, North Oaks, MN (US); James J. Rohl, Prscott, WI (US); Eric S. Edell, Rochester, MN (US); John J. Mullon, Rochester, MN (US); James P. Utz, Rochester, MN (US); Karen L. Swanson, Rochester, MN (US); Fabien Maldonado, Rochester, MN (US); Holly Kimball, Maple Grove, MN (US); Charles Rundquist, White Bear Lake, MN (US); Brian Cornwell, Big Lake, MN (US); David J. Lehse, Oakdale, MN (US); Daniel Shuey, Spencer, IN (US); David E. Midthun, Oronoco, MN (US)

(21) Appl. No.: 14/540,251

(22) Filed: Nov. 13, 2014

Related U.S. Application Data
(60) Provisional application No. 61/904,824, filed on Nov. 15, 2013.
(60) Provisional application No. 62/067,139, filed on Oct. 22, 2014.

(51) Int. Cl. A61B 10/04; A61B 10/00

(52) U.S. Cl. CPC A61B 10/04; A61B 10/0096; A61B 2010/0208
USPC 600/567

(57) ABSTRACT

This document provides medical device systems and methods for obtaining tissue samples. For example, this document provides medical device systems and methods for transbronchial needle biopsy tissue acquisition. In some cases, a needle biopsy system includes an actuator device, an outer needle with a lumen therethrough, and an inner needle at least partially disposed within the lumen. The outer needle can extend distally from the actuator device. A distal tip of the inner needle can be capable of being fully disposed within the lumen. The inner needle can extend distally from the actuator device. The actuator device can be configured to translate the outer needle proximally and distally. The actuator device can be configured to translate the inner needle proximally and distally independently of the outer needle.

### CLAIM OF PRIORITY

This application claims priority to U.S. Provisional Application Ser. No. 61/904,824, filed on Nov. 15, 2013 and to U.S. Provisional Application Ser. No. 62/067,139 filed Oct. 22, 2014, the entire contents of which are hereby incorporated by reference.

### BACKGROUND

1. Technical Field

This document relates to medical device systems and methods for obtaining tissue samples. For example, this document relates to medical device systems and methods for transbronchial needle biopsy tissue acquisition.

2. Background Information

A needle biopsy is a medical procedure for obtaining a sample of cells from a body for laboratory testing. Common needle biopsy procedures include fine-needle aspiration (FNA) and core needle biopsy. Needle biopsy may be used to take tissue or fluid samples from muscles, lymph nodes, bones, and organs such as the liver or lungs. A needle biopsy procedure is safer and less traumatic than an open surgical biopsy.

In some circumstances, FNA devices are used in pulmonary applications to sample lymph node tissues. Lymph node tissue samples are then examined to diagnose conditions such as mediastinal and hilar lymphadenopathy, lung cancer, and for staging lung cancer. Although the numbers of biopsy procedures are growing, there are several unmet clinical needs with current FNA needle designs. For example, one challenge of lymph node FNA is to sample sufficient core tissue volume from the relevant lymph nodes for accurate disease diagnosis and staging, while providing adequate material for ancillary testing such as mutation testing when indicated.

In one example FNA biopsy procedure, an endobronchial ultrasound (EBUS-guided) FNA biopsy of lung and lymph node tissue is performed using a flexible bronchoscope and localized anesthesia. EBUS uses real-time ultrasound technology to precisely locate the patient's lymph nodes, which improves the tissue collection for diagnostic purposes and reduces procedural risk.

EBUS-guided lymph node biopsy procedures are performed by first identifying a target lymph node using the ultrasound capability of the EBUS bronchoscope. Then, as the EBUS bronchoscope is held in a fixed location, a catheter containing a biopsy needle is advanced through a channel in the bronchoscope towards the target lymph node. After the needle has penetrated the lymph node, suction is applied through a lumen of the needle to trap the lymph node tissue within the lumen of the needle. Additional “jabs” are then made in which the biopsy needle is partially withdrawn from the lymph node and reinserted multiple times without fully withdrawing the biopsy needle from the lymph node. The biopsy needle is then removed from the bronchoscope. Finally, positive pressure is applied to the biopsy needle lumen to push the tissue sample out of the biopsy needle and into a sample collection container for histological examination and disease diagnosis.

### SUMMARY

This document provides medical device systems and methods for obtaining tissue samples. Medical device systems and methods provided herein can be used to obtain and/or biopsy tissue samples collected throughout the body (e.g., from the GI track, from organs, etc.). For example, this document provides medical device systems and methods for transbronchial needle biopsy tissue acquisition.

In a first general aspect, this document features a needle biopsy system. The needle biopsy system comprises an actuator device, an outer needle with a lumen therethrough, and an inner needle at least partially disposed within the lumen. The outer needle extends distally from the actuator device. The inner needle also extends distally from the actuator device. A distal tip of the inner needle is capable of being fully disposed within the lumen of the outer needle. The actuator device is configured to translate the outer needle proximally and distally. The actuator device is also configured to translate the inner needle proximally and distally, and to do so independently of the outer needle.

In various implementations of the needle biopsy system, the actuator device may be configured to rotate the inner needle as the actuator device translates the inner needle. The inner needle may optionally include a distal end portion with a spiral configuration. In some cases, the inner needle includes a distal end portion with interstitial spaces that are configured to retain tissue material. In some cases, the actuator includes an outer needle drive motor and an inner needle drive motor, and the outer needle drive motor and the inner needle drive motor are not the same, i.e., separate, motors. The actuator may optionally include a power source that supplies electrical current to the outer needle drive motor and the inner needle drive motor. In some cases, a spatial relationship exists between the inner needle and the outer needle that is configured for shearing tissue therebetween. In some cases, the outer needle comprises a tubular body defining a plurality of slots. In some examples, the distal tip of the inner needle is positioned within the lumen of the outer needle in a first arrangement, and the distal tip of the inner needle is positioned out of the lumen of the outer needle in a second arrangement. In some cases, the system is configured to move the outer and inner needles between a first and second arrangements by actuating the outer needle drive motor and the inner needle drive motor. The inner needle may include a distal end portion with a spiral configuration. In some cases, the inner needle includes a distal end portion with interstitial spaces that are configured to retain tissue material. In some embodiments, the inner needle has a main body portion and a tapered distal portion located proximal to the distal end portion of the inner needle, the tapered distal portion having a smaller diameter than a main body portion. A spatial relationship between the outer needle and the inner needle can be configured for shearing tissue therebetween.

In a second general aspect, this document features a method of collecting a tissue sample using a needle biopsy system comprising an actuator, an outer needle and an inner needle. The method may include inserting the needle biopsy system into a tissue collection apparatus; inserting the needle biopsy system through the septum into the interior cavity; retracting the outer needle, using the actuator, from the interior cavity and exposing a tissue sample disposed over the outer surface of the inner needle; and retracting the inner needle, using the actuator, from the interior cavity and engaging the septum with the tissue sample such that the tissue sample remains within the interior cavity of the vial. The tissue collection apparatus may include a vial, a flexible septum and an interior cavity configured to receive a biological sample. The interior cavity may be defined by a closed end portion of the vial and the flexible septum disposed within an interior portion of the vial.

In various implementations of the method, the needle biopsy system is retracted through the septum that is self-sealing such after the needle biopsy passes through the septum the septum is able to retain any liquids or biological tissue sealed within the interior cavity. In some cases, the needle biopsy system is inserted or retracted through the septum that comprises a slit formation, the slit formation defining a plurality of flaps configured to deflect outwardly or inwardly to create an opening for the needle biopsy passing through the septum. In some embodiments, the needle biopsy system is inserted through the septum that is sized and shaped complementary to the interior cavity of the vial.

In a third general aspect, this document features a method of obtaining a tissue sample from a subject. The method may include installing a bronchoscope into the subject; identifying (using the bronchoscope) a tissue area from which to obtain the tissue sample; installing a needle biopsy system into a channel of the bronchoscope; positioning a distal tip of the outer needle and a distal tip of the inner needle adjacent to the tissue area; advancing (using the actuator) the inner needle into the tissue area, wherein the inner needle simultaneously translates distally and rotates while advancing; advancing (using the actuator) the outer needle into the tissue area such that the distal tip of the inner needle is within the lumen of the outer needle, wherein advancing the outer needle shears tissue between the inner and outer needles such that the tissue sample is disposed within the lumen of the outer needle; and withdrawing the needle biopsy system from the bronchoscope. The needle biopsy system can include an actuator, an outer needle, and an inner needle. The inner needle can be at least partially within a lumen of the outer needle.

In various implementations of the method of obtaining a tissue sample from a subject, the method may further comprise extracting the tissue sample by advancing (using the actuator) the inner needle to remove the tissue sample from the lumen of the outer needle.

Particular cases of the subject matter described in this document can be implemented to realize one or more of the following advantages. First, using the systems and methods provided herein, a desired tissue sample volume can be obtained with a single biopsy needle penetration. Second, in comparison to some current biopsy methods, an increased volume of tissue can be sampled. Third, the tissue architecture of tissue samples can be substantially maintained using the sampling systems and methods provided herein. Therefore, better diagnosis of some types of cancer (e.g., lymphomas) can be attained. Fourth, the systems and methods provided herein enable controlled insertion depth into tissue, and without loss of ultrasonic visualization. For example, lymph node migration during node contact and penetration is reduced in comparison to current needle biopsy methods. Fifth, the operation of the biopsy system is efficient and convenient because of automated actuation by which the biopsy needle is inserted into the tissue. Sixth, the systems and methods provided herein can improve patient disease diagnosis, and reduce procedural time and cost by enabling more consistent sample collection and by reducing procedure complexity.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more cases of the invention are set forth in the accompanying drawings and the description herein. Other features, objects, and advantages of the invention will be apparent from the description and drawings, and from the claims.

### DESCRIPTION OF THE DRAWINGS

FIG. 1 is an illustration of a patient undergoing an EBUS-guided lymph node biopsy procedure in accordance with some cases provided herein.

FIG. 2 is a perspective view of the distal tip portion of an EBUS bronchoscope that is transmitting a needle biopsy device in accordance with some cases provided herein.

FIG. 3 is a perspective view of the distal tip portion of a needle biopsy system in accordance with some cases provided herein.

FIG. 4 is a side view of the distal tip portion of another needle biopsy system in accordance with some cases provided herein.

FIG. 5A is a side view of an exemplary outer needle tissue sampling member for use in the needle biopsy system provided herein.

FIGS. 5B-C are cross-sectional views of outer needle tissue sampling member of FIG. 5A.

FIGS. 6A-6C are side views of example inner needle tissue sampling members for use in the needle biopsy systems provided herein.

FIGS. 7A-C are side views of example inner and outer needle tissue sampling members for use needle biopsy system in accordance with some cases provided herein.

FIGS. 8A-8C are perspective views of the distal tip portions of additional needle biopsy systems in accordance with some cases provided herein.

FIG. 9 is a perspective view showing the top of a needle biopsy actuator in accordance with some cases provided herein.

FIG. 10 is a perspective view showing the bottom of the needle biopsy actuator of FIG. 9.

FIG. 11 is an exploded view of the needle biopsy actuator of FIG. 9.

FIGS. 12A-12E are a series of illustrations demonstrating the functioning of the needle biopsy actuator of FIG. 9 during a tissue sampling procedure in accordance with some cases provided herein.

FIG. 13 is a perspective view of another example needle biopsy actuator in accordance with some cases provided herein.

FIG. 14 is side view of the needle biopsy actuator of FIG. 13.

FIG. 15 is cross-sectional view of the needle biopsy actuator of FIG. 13.

FIG. 16 is an illustration of an exemplary tissue collection assembly in accordance with some cases provided herein.

FIG. 17 is a perspective view of the tissue collection assembly of FIG. 16.

FIG. 18 is a top view of an exemplary septum in accordance with some cases provided herein.

FIG. 19 is a flowchart of a method for performing a tissue biopsy procedure using a needle biopsy system in accordance with some cases provided herein.

FIG. 20 is a flowchart of a method for extracting tissue sample material from a needle biopsy system in accordance with some cases provided herein.

Like reference numbers represent corresponding parts throughout.

### DETAILED DESCRIPTION

This document provides medical device systems and methods for obtaining tissue samples. For example, this document provides medical device systems and methods for transbronchial needle biopsy tissue acquisition. While the systems and methods provided herein are described in the context of a transbronchial needle biopsy implementation, other implementations are also envisioned. For example, the systems and methods provided herein may also be advantageously applied for obtaining tissue samples in the GI tract, liver, pancreas, brain, kidneys, testicles, colon, thyroid, bladder, bone, cartilage, bladder, breast, uterus, stomach, heart, lungs and other locations within the body.

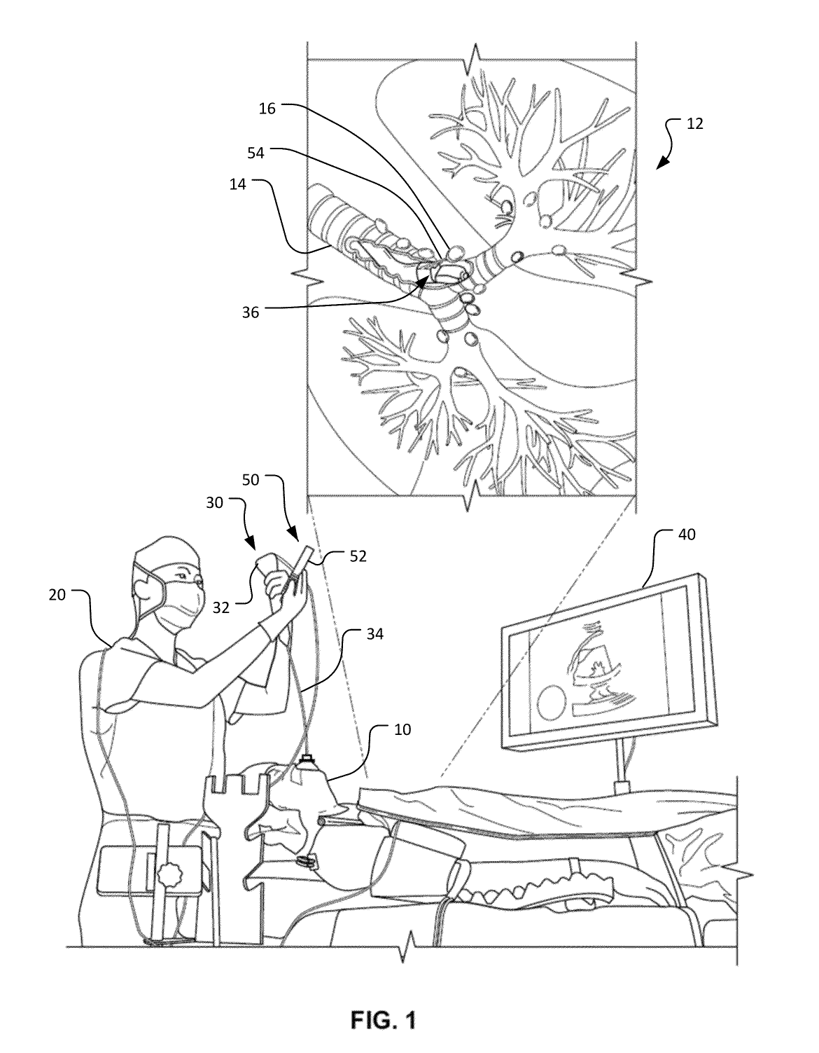

Referring to FIG. 1, an EBUS-guided lymph node biopsy procedure can be performed on a patient 10 by a clinician 20 using the systems and methods provided herein. For example, clinician 20 can use an EBUS bronchoscope 30 that is connected to a video monitor 40 to partially visualize an internal thoracic region 12 of patient 10. When clinician 20 has positioned a distal end portion 36 of EBUS bronchoscope 30 in a desired position in relation to a target tissue such as a lymph node 16, clinician 20 can activate a needle biopsy system 50 to collect a tissue sample. Thereafter, needle biopsy system 50 can be removed from EBUS bronchoscope 30 and the tissue sample material can be extracted from needle biopsy system 50 for laboratory analysis.

Internal thoracic region 12 of patient 10 is schematically represented in the upper frame of FIG. 1 for ease of understanding the systems and methods provided herein. In addition, a trachea 14 of patient 10 is depicted therein in a partially cut-away view so that distal end portion 36 of EBUS bronchoscope 30 within trachea 14 can be visualized.

EBUS bronchoscope 30, in brief, can include a handle 32, a flexible probe 34, and distal end portion 36. Handle 32 is coupled to flexible probe 34, and flexible probe 34 extends distally from handle 32 and terminates at distal end portion 36. Clinician 20 can manipulate and operate EBUS bronchoscope 30 using handle 32. For example, clinician 20 can navigate flexible probe 34 through the airways (mouth, nose, pharynx, larynx, trachea, bronchi branches, etc.) of patient 10 as desired by manipulating handle 32. In some cases, clinician 20 can perform other operations using handle 32, including switching between fiber optic and ultrasonic viewing modalities, rotating or pivoting the ultrasonic array, and other operations.

Video monitor 40 can receive image data from EBUS bronchoscope 30 and display the image data for viewing by clinician 20. In this fashion, clinician 20 can visualize the navigation and positioning of distal end portion 36 of flexible probe 34 within the airway of patient 10. Clinician 20 can thereby navigate distal end portion 36 to a target tissue area from which a tissue sample is desired. For instance, in this example clinician 20 is navigating bronchoscope distal end portion 36 through trachea 14 so as to locate a target lymph node 16 that clinician 20 desires to obtain a tissue sample from.

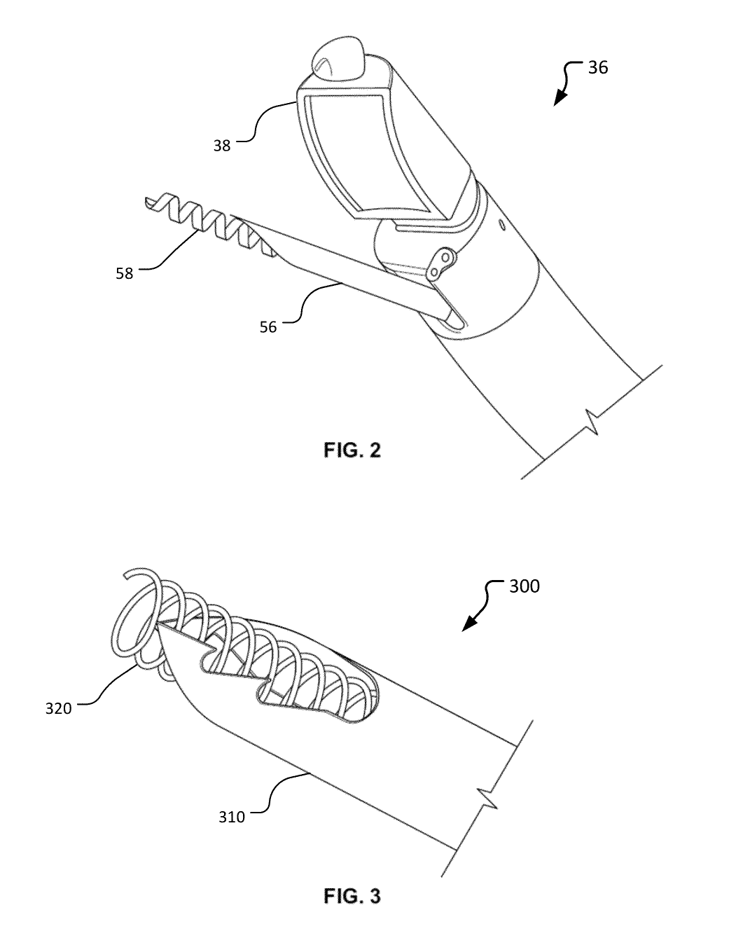

Referring now to FIGS. 1 and 2, needle biopsy system 50 includes an actuator 52, a sheath 54 (which may also be referred to as a catheter), an outer needle 56, and an inner needle 58. In FIG. 2, sheath 54 is omitted to enable better visualization of outer needle 56 and inner needle 58 which are shown as having been actuated to extended configurations (as will be described further below). Sheath 54, outer needle 56, and inner needle 58 each extend distally from actuator 52. Sheath 54 and outer needle 56 are each tubular. Inner needle 58 is at least partially located within the tubular interior of outer needle 56. Outer needle 56, in turn, is at least partially located within the tubular interior of sheath 54.

In some cases, sheath 54 (containing outer needle 56 and inner needle 58) is threaded into a lumen (e.g., an instrument channel) within flexible probe 34 of EBUS bronchoscope 30, and actuator 52 is then releasably coupled to bronchoscope handle 32 for convenient use by clinician 20. The axial lengths of sheath 54, outer needle 56, and inner needle 58 can allow the distal end portions thereof to extend from distal end portion 36 of flexible probe 34 in some configurations (as illustrated in FIG. 2) so as to penetrate the tissue area to be sampled. This extension is at least partially the result of activation by clinician 20 of motors in needle biopsy system 50, as will be described further below. As outer needle 56 and inner needle 58 are activated to extend from distal end portion 36 of flexible probe 34, trachea 14 is pierced and target lymph node 16 is penetrated first by inner needle 58 and then by outer needle 56, and a sample of lymph node 16 is obtained as will be described further below.

Sheath 54 can comprise a tubular polymeric or metallic material. For example, in some cases, sheath 54 can be made from polymeric materials such as, but not limited to, polytetrafluoroethylene (PTFE), fluorinated ethylene propylene (FEP), Hytrel®, nylon, Picoflex®, Pebax®, and the like. In alternative cases, sheath 54 can be made from metallic materials such as, but not limited to, nitinol, stainless steel, stainless steel alloys, titanium, titanium alloys, and the like.

Outer needle 56 can comprise a tubular metallic material. For example, in some cases, outer needle 56 can be made from metallic materials such as, but not limited to, nitinol, stainless steel, stainless steel alloys, titanium, titanium alloys, and the like. Outer needle 56 can be made in a variety of sizes to suit different applications. For example, in some cases, a 19 gauge hypo tubing material is used to make outer needle 56. In some cases, a 22 gauge, 25 gauge or 27 gauge hypo tubing material is used to make outer needle 56. Other larger or smaller sizes of tubing materials may also be used in some implementations. Outer needle 56 can be made with various wall thicknesses. For example, in some cases, outer needle 56 can have a wall thickness in the range of about 0.002 inches to about 0.006 inches (about 0.05 millimeters to about 0.2 millimeters). As shown in FIG. 2, the distal end portion of outer needle 56 can be beveled to create a sharp tip for penetrating tissue as outer needle 56 is axially translated in a distal direction. The tip's sharpness can advantageously enhance the ability of needle biopsy system 50 to collect a tissue sample while substantially maintaining the cellular architecture of the tissue. While outer needle 56 has a single-angle beveled distal end portion, other outer needle cases can include other styles of sharp distal end portions.

Inner needle 58 can comprise a polymeric, metallic, or composite material. For example, in some cases, inner needle 56 can be made from metallic materials such as, but not limited to, nitinol, stainless steel, stainless steel alloys, titanium, titanium alloys, platinum, composite materials, and the like. The size of the outer diameter of inner needle 58 can be selected to complement or correspond to the size of the inner diameter of outer needle 56. In some cases, a clearance therebetween of about 0.0005 inches (about 0.013 millimeters) per side is desirable. In some cases, a clearance therebetween in a range of about 0.000 inches to about 0.001 inches (about 0.000 millimeters to about 0.0254 millimeters) per side is desirable. In some cases, a clearance therebetween in a range of about 0.0005 inches to about 0.002 inches (about 0.013 millimeters to about 0.051 millimeters) per side is desirable. In some cases, a clearance therebetween in a range of about 0.001 inches to about 0.004 inches (about 0.025 millimeters to about 0.102 millimeters) per side is desirable. Inner needle 58 can be made with various wall thicknesses. For example, in some cases, inner needle 58 can have a wall thickness in the range of about 0.002 inches to about 0.006 inches (about 0.05 millimeters to about 0.2 millimeters).

In various embodiments, outer diameter of inner needle 58 and inner diameter of outer needle 56 can be selected to obtain a suitable clearance therebetween. For example, in some cases, outer needle can have an outer diameter of about 0.044 inches (about 1.1 millimeters) and inner diameter of about 0.0345 inches (about 0.876 millimeters), and inner needle can have an outer diameter of 0.0340 inches (about 0.864 millimeters). In another example, outer needle can have an outer diameter of about 0.0275 inches (about 0.699 millimeters) and inner diameter in the range of about 0.0215 inches to about 0.0220 inches (about 0.546 millimeters to about 0.559 millimeters), and inner needle can have an outer diameter of about 0.021 inches (about 0.53 millimeters). In yet another example, outer needle can have an outer diameter of about 0.021 inches (about 0.53 millimeters) and inner diameter in the range of about 0.0115 inches to about 0.012 inches (about 0.292 millimeters to about 0.31 millimeters), and inner needle can have an outer diameter of about 0.011 inches (about 0.28 millimeters).

The distal end portion of inner needle 58 has a generally spiral shape. In some cases, while the distal end portion of inner needle 58 is spirally shaped, the portions of inner needle that are proximal of the distal end portion comprise a flexible cylindrical shaft member. In particular cases, the distal end portion of inner needle 58 is a generally helically-shaped spiral. Such spiral shapes facilitate the penetration of inner needle 58 into tissue as inner needle 58 is simultaneously rotated and translated axially, as will be explained further below. In addition, the interstitial space between the spirals allows tissue material to accumulate and be retained therein, thereby collecting sample tissue material in the needle biopsy system.

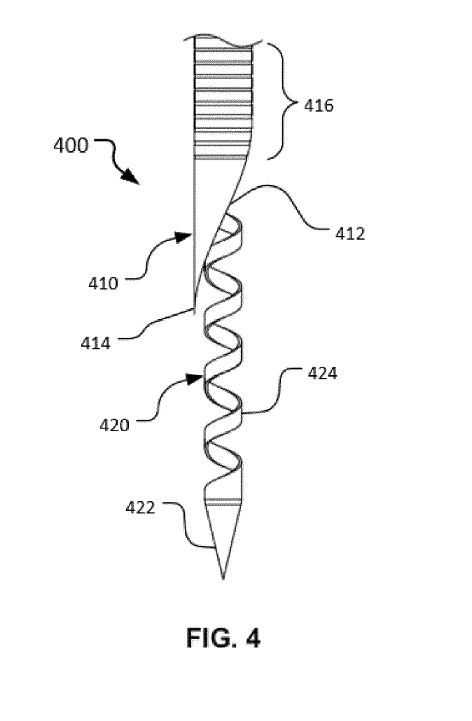

Referring to FIG. 3, in some cases, the distal end portion 300 of a needle biopsy system includes an outer needle 310 and an inner needle 320 that have unique designs for penetrating and shearing tissue. In this embodiment, the distal end portion of outer needle 310 is configured with dual penetrating tips and serrated edges. Such a configuration can, for example, enhance penetration and shearing of tissue while substantially maintaining the cellular architecture of the tissue. Outer needle 310 illustrates that a wide variety of configurations of outer needle distal end portions are envisioned within the scope of this disclosure.

In this embodiment, inner needle 320 has a distal end portion that is configured as a coil. The very distal tip of the coil can be a sharp point for facilitating penetration of tissue. In use, inner needle 320 simultaneously rotates and translates distally with a screw-like motion. The rotation and translational motion of inner needle 320 can substantially match the pitch of the coil of inner needle 320. Therefore, the very distal tip of the coil shears the tissue in a substantially uniform helical path as the coil penetrates the tissue. That helical path substantially matches the coil's shape. This configuration of inner needle 320, and the motion thereof, can thereby enhance penetration and shearing of tissue while substantially maintaining the cellular architecture of the tissue.

Still referring to FIG. 3, the general operation of the needle biopsy systems provided herein will now be briefly explained using distal end portion 300 as an example. First, distal end portion 300 is positioned adjacent to and in axial alignment with a target tissue area. Inner needle 320 is then advanced distally to penetrate into the target tissue. The motion of inner needle 320 is both rotational and translational. While inner needle 320 is advancing, the position of outer needle 310 is maintained substantially stationary. After inner needle 320 has been advanced into the target tissue to a desired depth, the motion of inner needle 320 is stopped. Next, outer needle 310 is advanced distally. The motion of outer needle 310 is translational. As outer needle 310 translates, outer needle 310 encapsulates inner needle 320. In addition, as outer needle 310 translates, the tissue between the outer diameter of inner needle 320 and the inner diameter of outer needle 310 is sheared. After such shearing, an amount of tissue remains within the coils of inner needle 320. The tissue within the turns of the coil of inner needle 320 is captured therein because outer needle 310 has encapsulated inner needle 320. In this configuration, the needles 310 and 320 can then be removed (pulled back) from the target tissue and fully out of the channel of the bronchoscope, while the tissue material remains within the coils of inner needle 320. To eject the sample tissue material, inner needle 320 can be advanced distally in relation to outer needle 310 to expose the tissue material contained within the coils of inner needle 320. The sample tissue material can thereafter be removed from the coils of inner needle 320.

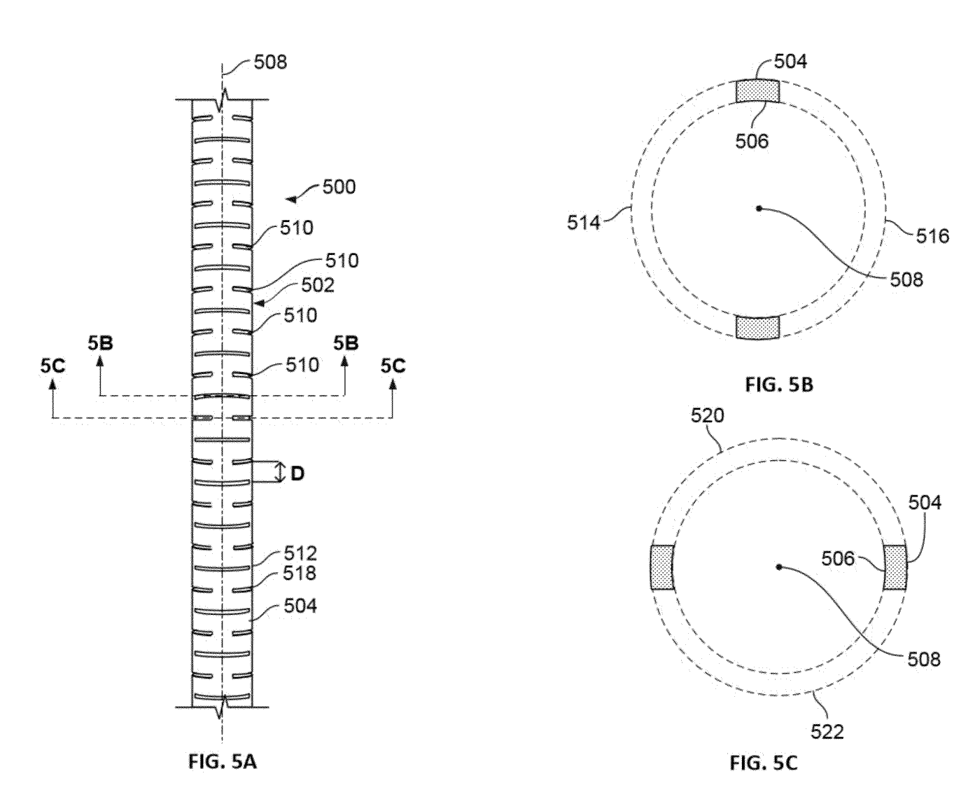

Referring to FIG. 4, example distal end portion 400 can be used in some cases, of the needle biopsy systems provided herein. Distal end portion 400 includes an outer needle 410 and an inner needle 420. Outer needle 410 is configured with a single bevel 412 and a sharp tip 414. In addition, outer needle 410 has example echogenic features 416 that can enhance the visibility of outer needle 410 under ultrasound. Such echogenic features of various types can be included on any of the needles (both outer and inner) provided herein.

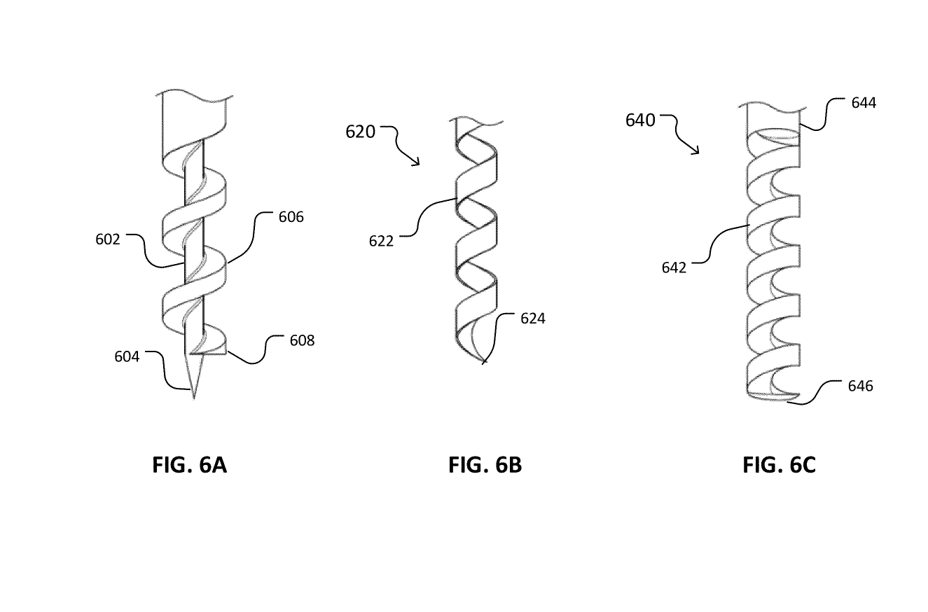

Referring to FIGS. 5A-5C, an exemplary outer needle 500 can be used in some cases of the needle biopsy systems provided herein. As shown, outer needle 500 includes an elongate tubular body 502 with an outer diameter 504, an inner diameter 506. Tubular body 502 can define a central axis 508. At least a portion of tubular body 502, as shown in FIG. 5A, defines a plurality of slots 510 longitudinally along tubular body 502.

Still referring to FIGS. 5A-5C, each slot of the plurality of slots 510 is defined by a suitable width and an arc length that extends perpendicular to central axis 508 and along a portion of a circumference of tubular body 502. Each slot of the plurality of slots 510, as shown in FIG. 5A, extends at an angle perpendicular to central axis 508 of tubular body 502. In some cases, each slot can extend at an angle perpendicular or oblique to central axis 508. In FIG. 5A, each slot extends at an angle that is parallel to any adjacent slots. In some cases, however, a slot can extend in an angle that is different from adjacent slots. Each slot can be longitudinally spaced a suitable distance from an adjacent slot by a separation distance D. In some cases, separation distance D between a slot and an adjacent slot can be uniform and constant while, in other cases, separation distance D between a slot and an adjacent slot can be varied. For example, in some cases, separation distance D between a slot and an adjacent slot is about 0.0075 (about 0.19 millimeters) or about 0.015 inches (about 0.38 millimeters). Generally, separation distance D between each slot and an adjacent slot can be any value in the range of about 0.0070 inches to about 0.015 inches (about 0.18 millimeters to about 0.38 millimeters), about 0.015 inches to about 0.025 inches (about 0.38 millimeters to about 0.64 millimeters), or about 0.025 inches to about 0.050 inches (about 0.64 millimeters to about 1.3 millimeters). Arc length, width and angle of each slot may be adjusted to achieve a suitable flexibility of outer needle 500.

Each slot of the plurality of slots 510 can have various dimensions at various longitudinal locations along tubular body 502. Dimensions of each slot of the plurality of slots 510 can be configured such that outer needle 500 has suitable functional strength for accessing a particular anatomy. In some cases, dimensions and locations of each slot of the plurality of slots 510 can minimize the amount of push force required when advancing needle biopsy system through a scope within a particular anatomy. For example, in some cases, a slot of the plurality of slots 510 has a width of about 0.001 inches (about 0.03 millimeters). In some cases, a slot of the plurality of slots 510 can have a width of any value in the range of about 0.0005 inches to about 0.0020 inches (about 0.01 millimeters to about 0.051 millimeters).

Still referring to FIGS. 5A-5C, at least a portion of the tubular body 502 of outer needle 500 includes a plurality of pairs of circumferentially opposing slots. Alternatively, in some cases, the outer needle 500 can include slots on one circumferential side of tubular body 502 to encourage preferential bending towards a particular direction. Referring to FIGS. 5A and 5B, a first pair of opposing slots 512 comprises a first slot 514 that is circumferentially opposite a second slot 516. Referring to FIGS. 5A and 5C, a second pair of opposing slots 518 is longitudinally adjacent to first pair of opposing slots 512, but rotated by a suitable degree of rotation. For example, as shown in FIG. 5B, second pair of opposing slots 518 is rotated 90 degrees relative to first pair of opposing slots 512. In some cases, second pair of opposing slots 518 can be rotated in relation to first pair of opposing slots 512 by 0, 30, 45, 60 or 90 degrees, or by any value within the range of 0 to 90 degrees. In some cases, at least a portion of the tubular body 502 of outer needle 500 includes a plurality of pairs of circumferentially opposing slots that are offset by a suitable distance. Although several arrangements are provided herein, various other arrangements of slots and pairs of slots are also possible.

Inner needle 420 includes a conical tip 422 and a coil-like working portion 424. In this configuration, conical tip 422 provides for efficient tissue penetration, such as for penetration of the patient's airway wall, for example. It should be understood, however, that all needle tip configurations provided herein are capable of providing appropriate tissue penetration performance. Coil-like working portion 424 provides interstitial space to collect sample tissue material.

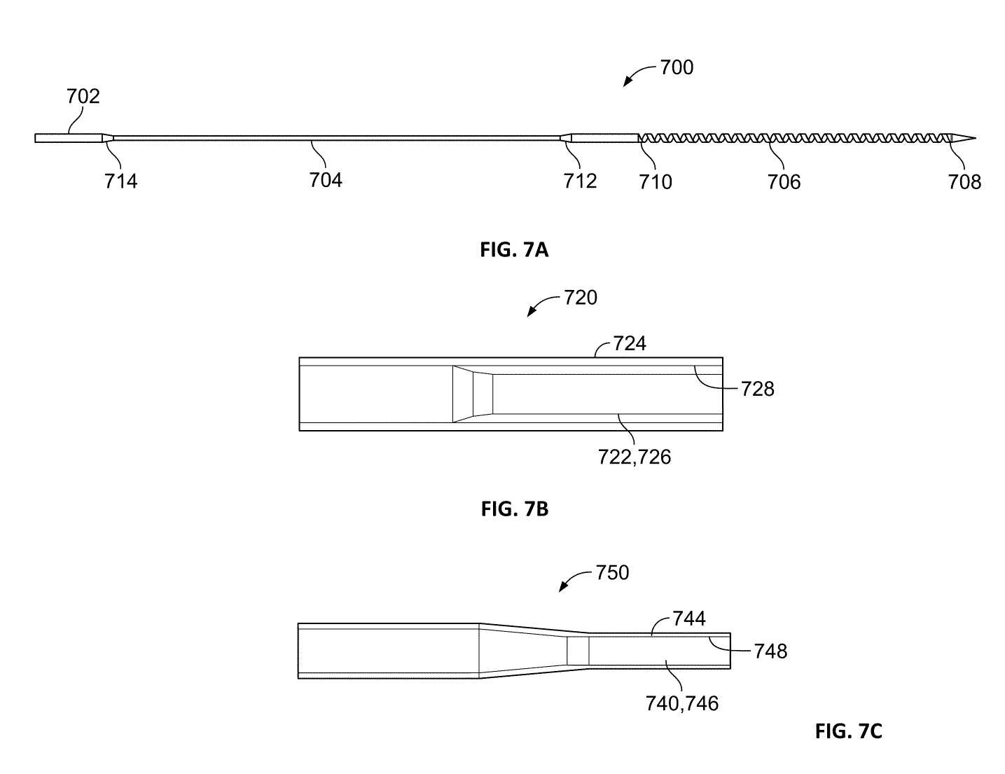

Referring to FIG. 6A, another example inner needle 600 is provided that can be used with some cases of the needle biopsy systems provided herein. This inner needle embodiment includes a central core member 602 that terminates at a conical distal tip 604. Around core member 602 is a spiral member 606 that terminates at a distal beveled distal edge 608. As inner needle 600 is advanced into a target tissue area, conical distal tip 604 acts as the leading portion to penetrate the tissue. In addition, as described previously, in use beveled distal edge 608 is rotating while inner needle 600 is translating distally. Beveled distal edge 608 thereby moves along a spiral path, and beveled distal edge 608 shears tissue along the spiral path. The sheared tissue material collects within the interstitial spaces of spiral member 606.

Referring to FIG. 6B, another example inner needle 620 is provided that can be used with some cases of the needle biopsy systems provided herein. This inner needle embodiment includes a coil member 622 that terminates at a pointed distal tip 624. In some cases, the edges of coil member 622 that define pointed distal tip 624 are sharpened to facilitate efficient tissue shearing. In addition, the very tip of pointed distal tip 624 can be a sharp point to facilitate efficient tissue shearing. Sheared tissue material collects within the interstitial spaces of coil member 622.

While the pitches of the spiral member of the inner needles provided herein are generally illustrated as uniform along the length of the spiral member, such pitch uniformity is not required. That is, in some cases, the inner needle may have an inconsistent pitch along the length of the spiral member, or at particular portions of the spiral member.

Referring to FIG. 6C, another example inner needle 640 is provided that can be used with some cases of the needle biopsy systems provided herein. This inner needle embodiment includes a flexible shaft 644 from which a coil member 642 extends distally. In this embodiment, in comparison to inner needle 620 for example, coil member 642 has a wider web extending towards the axis of inner needle 640. Coil member 642 terminates at a beveled distal edge 646. As inner needle 640 is advanced into a target tissue area, beveled distal edge 646 acts as the leading portion for penetrating the tissue. In addition, as described previously, beveled distal edge 646 is rotating while inner needle 640 is translating distally. Beveled distal edge 646 thereby moves along a spiral path, and beveled distal edge 646 shears tissue along the spiral path. The sheared tissue material collects within the interstitial spaces of coil member 642.

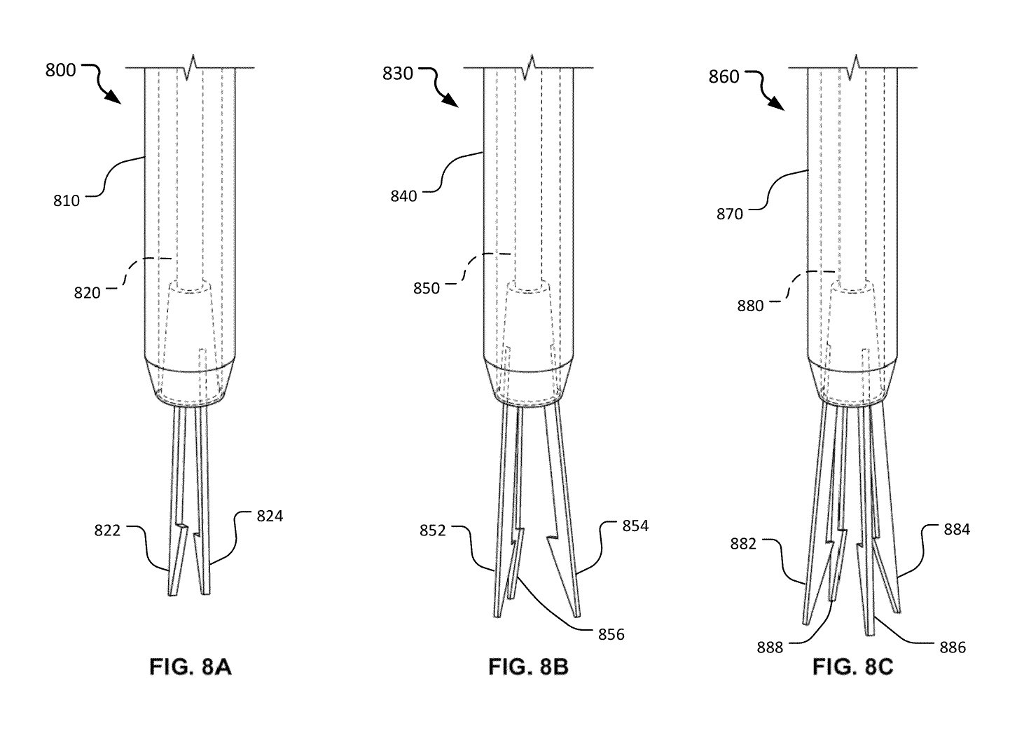

Referring to FIG. 7A, another exemplary inner needle 700 is provided that can be used with some cases of the needle biopsy systems provided herein. Inner needle 700 can include a main body 702 and a tapered portion 704 located proximate to a distal end portion 706. Distal end portion 706 of inner needle 700 can have a generally spiral shape that extends between a distal end 708 and a proximal end 710 of distal end portion 706.

In some cases, inner needle 700 has at least one tapered portion 704. In some cases, tapered portion 704 is located at a suitable location proximal to distal end portion 706 and distal to main body 702 of inner needle 700. In some cases, the location of tapered portion 704 can increase flexibility of inner needle 700 in a localized area such that inner needle flexibility can access a particular anatomy or be used with an ancillary medical device, such as a scope. For example, in some cases, tapered portion 704 is located about 3.15 inches (about 8 centimeters) from distal end portion 706 or a very distal tip of the coil. In another example, in some cases, tapered portion 704 is located about 7.09 inches (about 18 centimeters) from distal end portion 706 or very distal tip of the coil. The location of tapered portion 704 can be any value ranging from about 3.15 inches (about 8 centimeters) to about 7.09 inches (about 18 centimeters), about 7.09 inches (about 18 centimeters) to about 12 inches (about 30 centimeters), and of about 12 inches (about 30 centimeters) to about 24 inches (about 61 centimeters) from distal end portion 706 or very distal tip of the coil. In other cases, tapered portion 704 is located directly adjacent to proximal end 710 of distal end portion 706. As shown in FIG. 7A, tapered portion 704 of inner needle 700 can include an outer diameter extending between a distal end 712 and a proximal end 714 of tapered portion 704 that is smaller than an outer diameter of main body 702 of inner needle 700.

As shown in FIG. 7B, an exemplary tapered portion 720 of an inner needle 722 within an outer needle 724 is provided in accordance with some cases of the needle biopsy systems provided herein. As shown, tapered portion 720 of inner needle 722 is disposed within a constant diameter lumen 724 of outer needle 724, creating an increase in clearance provided herein between an outer diameter 726 of inner needle 722 and inner diameter 728 of outer needle 724. In some cases,

Alternatively, in some cases, as shown in FIG. 7C, outer diameters of another exemplary inner needle 740 and an outer needle 744 are both tapered to a smaller dimension. Both inner and outer needles 740, 744 can be tapered to minimize areas within a needle biopsy system having increased clearance between an outer diameter 746 of inner needle 740 and inner diameter 748 of outer needle 744. Such designs where inner needle 740 alone or where inner and outer needles 740, 744 are both tapered can help to increase the flexibility of the needle biopsy systems. Tapered portions 750 of inner and outer needle 740, 744 can facilitate increasing the flexibility and kink resistance of select portions of the needle biopsy systems. In some cases, tapered portions 750 can improve the compatibility between a needle biopsy system with other medical devices, such as an outer sheath or tubing.

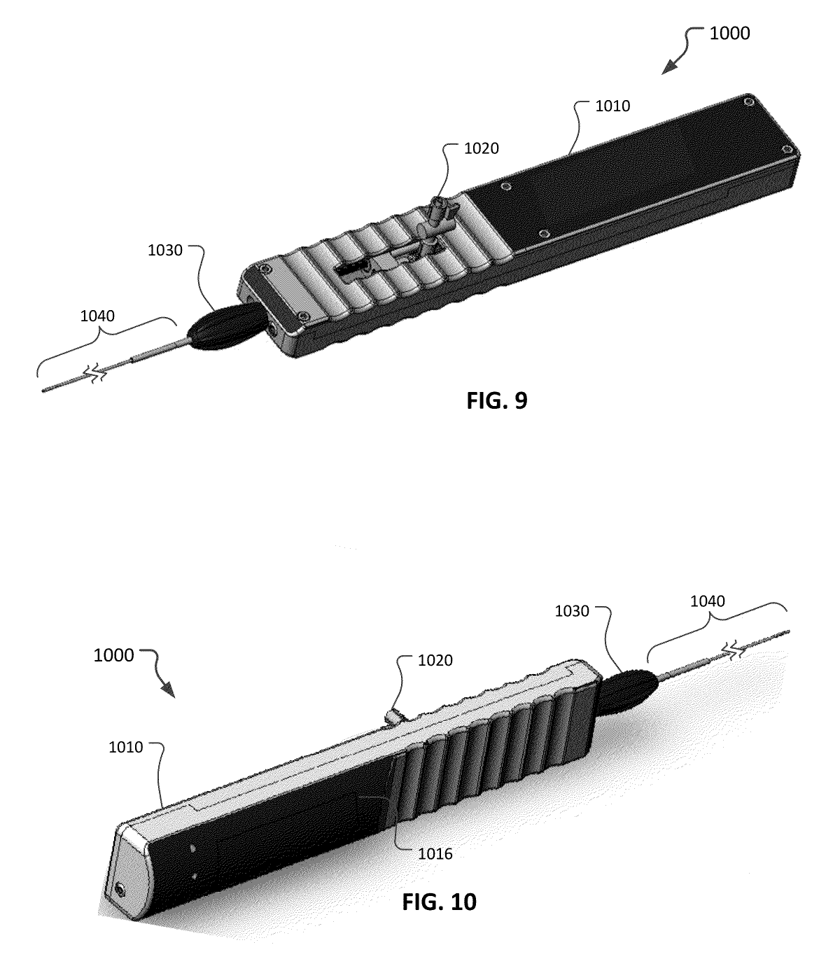

Referring to FIGS. 8A-8C, alternative distal end portions 800, 830, and 860 are provided that can be used with some cases of the needle biopsy systems provided herein. These cases operate in a different manner than the inner and outer needle cases described above (e.g., in reference to FIGS. 1-7). That is, while the motion of the inner needles of previously described cases included both rotational and translational aspects, the inner needles 820, 850, and 880 of distal end portions 800, 830, and 860 do not rotate.

Distal end portion 800 has an inner needle 820 with two barbed projections 822 and 824. Distal end portion 830 has an inner needle 850 with three barbed projections 852, 854, and 856. Distal end portion 860 has an inner needle 880 with four barbed projections 882, 884, 886, and 888.

Distal end portion 800 will be used to describe the operation of these cases (which are substantially similar to each other except for the number of barbed projections). As the needle biopsy system is moved into position near a target tissue, inner needle 820 is located within outer needle 810 such that the two barbed projections 822 and 824 are within the lumen of outer needle 810. At the target tissue site, inner needle 820 is translated distally such that barbed projections 822 and 824 emerge from outer needle 810 (e.g., as depicted in FIG. 8A) and pierce the target tissue. Outer needle 810 is then translated distally to re-encapsulate inner needle 820. In doing so, barbed projections 822 and 824 are forced towards each other to pinch tissue material such that the tissue material is sheared and captured within the confines between barbed projections 822 and 824, which are encapsulated within outer needle 810. Inner needle 820 and outer needle 810 are then removed as a unit from the patient. Lastly, the tissue sample contained within outer needle 810 can be ejected by extending inner needle 820 in relation to outer needle 810.

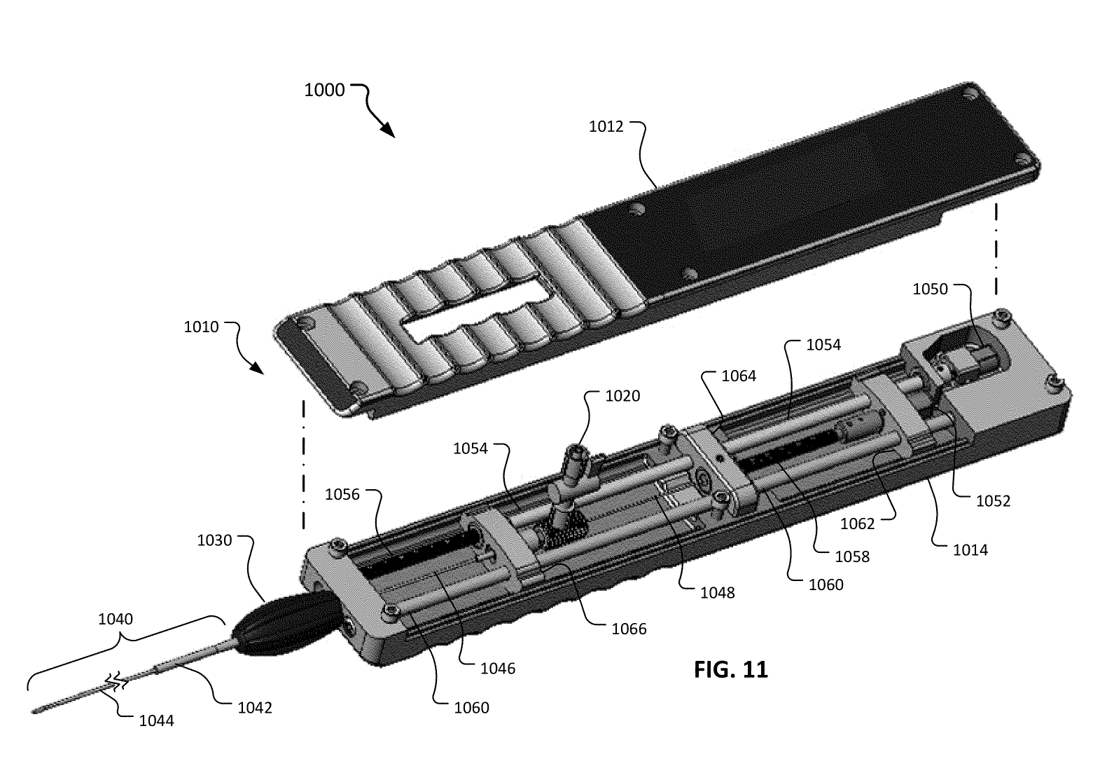

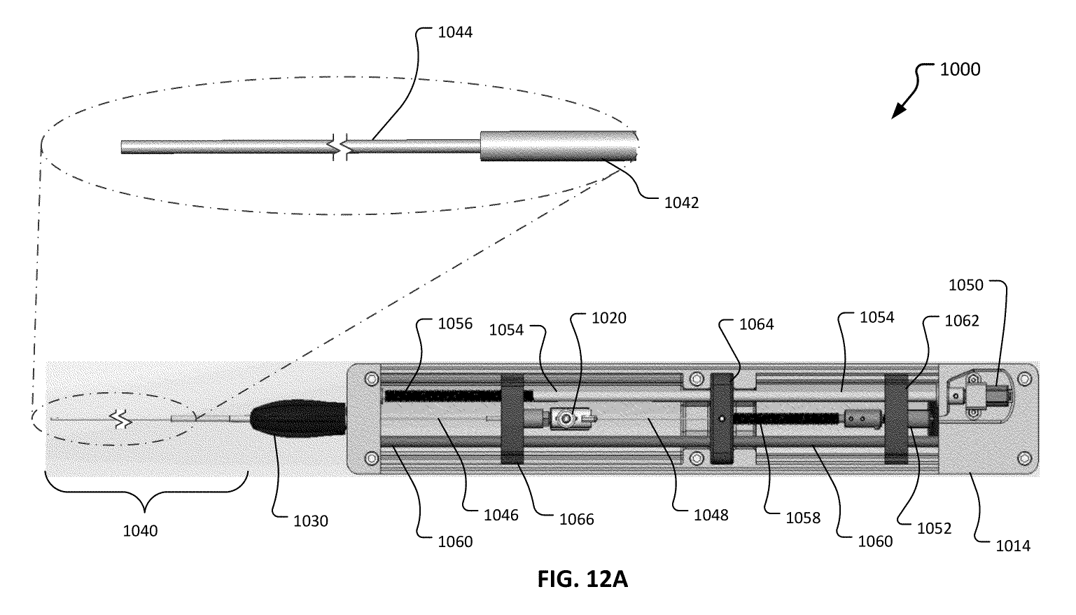

Referring to FIGS. 9 and 10, the needle biopsy system cases provided herein include an actuator such as example actuator 1000. Actuator 1000 can include a housing 1010, a suction stopcock 1020, a coupling member 1030, and a needle assembly 1040. Suction stopcock 1020 extends through a slot in housing 1010. Coupling member 1030 is attached to and extends from a distal end of housing 1010. Needle assembly 1040 extends from coupling member 1030.

In some cases, housing 1010 can be formed by machining or by mold processes. In particular cases, housing 1010 is an aluminum material that is machined to a configuration essentially as shown or similar thereto. In some cases, machined materials other than aluminum are used including, but not limited to, stainless steel, steel alloys, or various polymeric materials. In other cases, housing 1010 can be made from molded polymeric materials including, but not limited to, thermoplastics that include polymethyl methacrylate (PMMA or Acrylic), polystyrene (PS), acrylonitrile butadiene styrene (ABS), polyvinyl chloride (PVC), modified polyethylene terephthalate glycol (PETG), cellulose acetate butyrate (CAB); and semi-crystalline commodity plastics that include polyethylene (PE), high density polyethylene (HDPE), low density polyethylene (LDPE or LLDPE), polypropylene (PP), polymethylpentene (PMP), and the like.

In some cases, suction stopcock 1020 is a combination of a fitting and a valve. In some implementations, a negative pressure (i.e., vacuum, such as generated from a syringe) can be applied at suction stopcock 1020. In some such implementations, the negative pressure can be transmitted through the lumen of the outer needle, to the distal end thereof, and can assist with the collection of sample tissue by the needle biopsy system.

Coupling member 1030 can be configured to releasably couple actuator 1000 to a bronchoscope handle (e.g., refer to FIG. 1). The handle of a bronchoscope used in conjunction with actuator 1000 may have a corresponding complementary fitting that coupling member 1030 can mate with to releasably couple actuator 1000 to the handle.

In some cases, needle assembly 1040 includes an outer sheath, an outer needle, and an inner needle. The inner needle is at least partially within a lumen of the outer needle. The outer needle is at least partially within a lumen of the outer sheath. The component of needle assembly 1040 that is visible in FIGS. 9 and 10 is the outer sheath, and the outer and inner needles are within the outer sheath. Needle assembly 1040 is inserted into a lumen (e.g., instrument channel) of a bronchoscope in preparation for collecting a tissue sample.

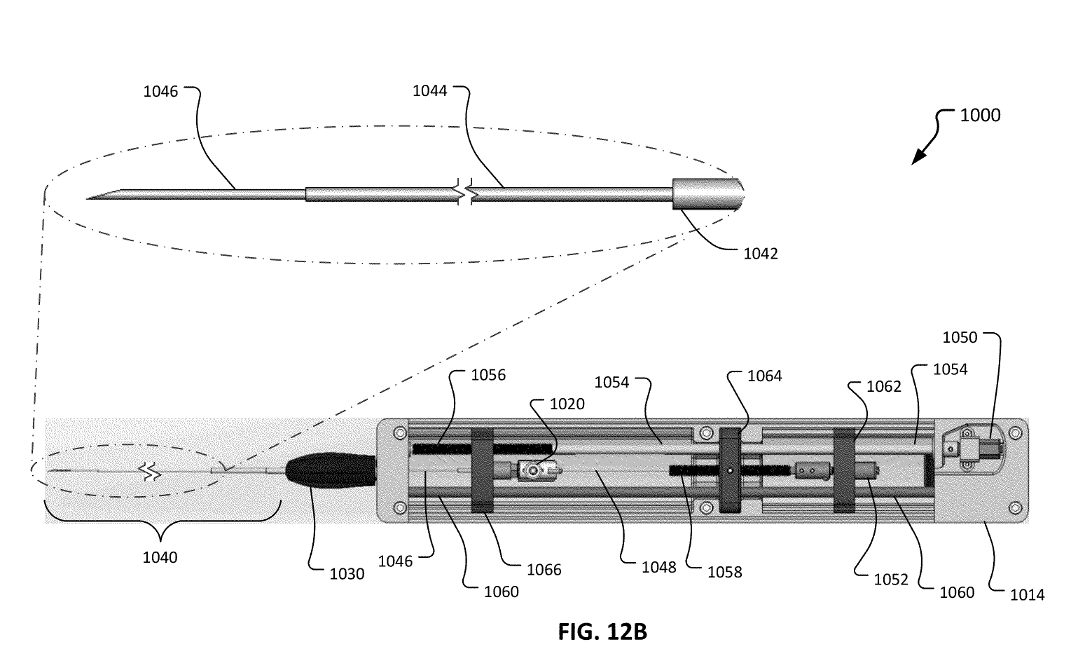

Referring to FIG. 11, a battery access panel 1016 is removably coupled to housing 1010. Battery access panel 1016 can be removed from housing 1010 to change one or more batteries that are located within housing 1010, and which provide electrical power for motors used in actuator 1000.

In FIG. 11, actuator 1000 is shown in an exploded view to facilitate visualization of the internal components associated with motion actuation. Housing 1010 includes a cover 1012 and a main housing body 1014. The components within main housing body 1014 include an outer needle 1046, an inner needle 1048, an outer needle drive motor 1050, an inner needle drive motor 1052, an outer needle drive shaft 1054, an outer needle lead screw 1056, an inner needle lead screw 1058, a stationary guide shaft 1060, an inner needle shuttle 1062, a stationary guide 1064, and an outer needle shuttle 1066. These components are included in this particular actuator 1000 embodiment, but are not required in all actuator cases envisioned within the scope of this disclosure. The interactions of the various components of example actuator 1000 will now be described.

Outer needle 1046 is coupled to outer needle shuttle 1066. Outer needle shuttle 1066 slides proximally and distally on stationary guide shaft 1060. Outer needle shuttle 1066 is coupled with outer needle lead screw 1056 in a threaded arrangement. That is, outer needle lead screw 1056 has an external thread and outer needle shuttle 1066 has a complementary internal thread. Therefore, as outer needle lead screw 1056 rotates, outer needle shuttle 1066 will be thereby driven to translate proximally or distally. As outer needle shuttle 1066 translates proximally or distally, outer needle 1046 also translates proximally or distally in a corresponding fashion. Outer needle lead screw 1056 is coupled to outer needle drive shaft 1054, which in turn is coupled to outer needle drive motor 1050. It can be understood, therefore, that actuation of outer needle drive motor 1050 will cause outer needle 1046 to translate distally or proximally (depending on the direction of rotation of outer needle drive motor 1050).

Inner needle 1048 is coupled to inner needle lead screw 1058. Therefore, as inner needle lead screw 1058 rotates, inner needle 1048 rotates in a corresponding fashion. Inner needle lead screw 1058 is coupled with stationary guide 1064 in a threaded arrangement. That is, inner needle lead screw 1058 has an external thread and stationary guide 1064 has a complementary internal thread. Stationary guide 1064 is held in a fixed position in relation to main housing body 1014. Therefore, as inner needle lead screw 1058 rotates, inner needle lead screw 1058 and inner needle 1048 translate proximally or distally in relation to housing 1010. Inner needle drive motor 1052 is coupled to inner needle lead screw 1058 at inner needle shuttle 1062. It can be understood, therefore, that actuation of inner needle drive motor 1052 will cause inner needle 1048 to both rotate and translate simultaneously.

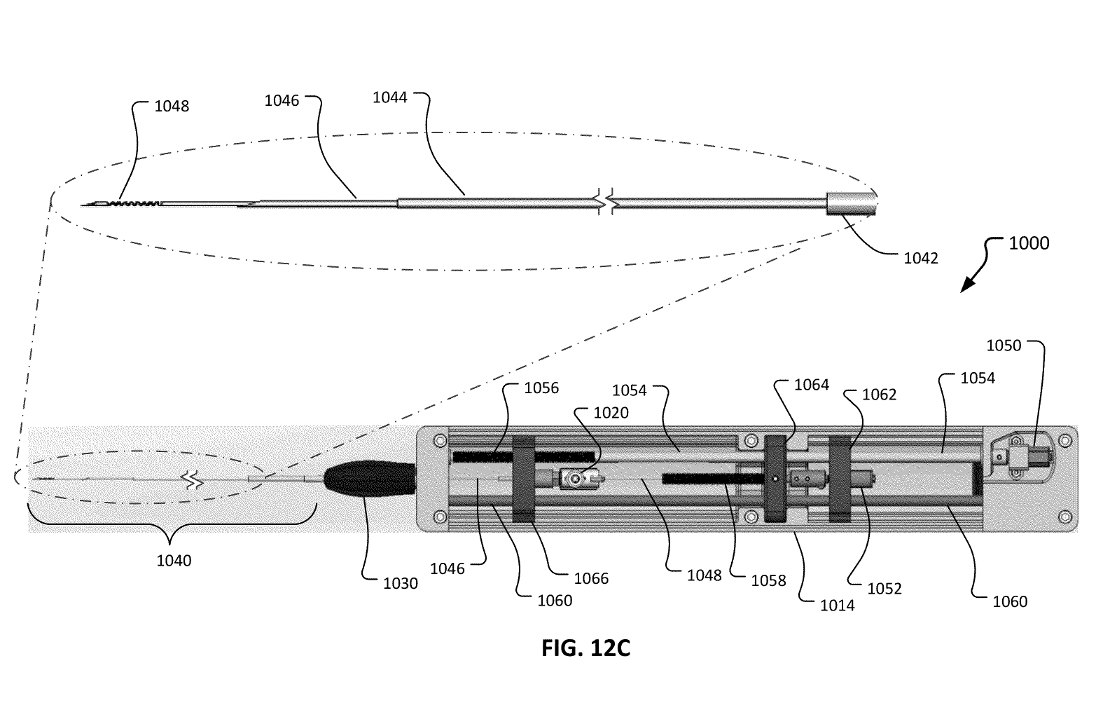

FIGS. 12A-12E are a series of sequential illustrations demonstrating the functionality of needle biopsy actuator 1000 during a tissue sampling procedure in accordance with some cases provided herein. FIG. 12A shows actuator 1000 in an arrangement having inner and outer needles 1048 and 1046 within sheath 1044. FIG. 12B shows actuator 1000 in an arrangement having inner and outer needles 1048 and 1046 extending distally out from the confines of sheath 1044. FIG. 12C shows actuator 1000 in an arrangement having inner and outer needles 1048 and 1046 extending distally out from the confines of sheath 1044, and inner needle 1048 further extending distally out from the confines of outer needle 1046. FIG. 12D shows actuator 1000 in an arrangement having inner and outer needles 1048 and 1046 extending distally out from the confines of sheath 1044, and outer needle 1046 extended to re-encapsulate inner needle 1048. FIG. 12E shows actuator 1000 in an arrangement having inner and outer needles 1048 and 1046 re-encapsulated within sheath 1044 as in the arrangement of FIG. 12A.

In FIG. 12A, outer needle 1046 and inner needle 1048 are in fully retracted positions such that outer needle 1046 and inner needle 1048 are encapsulated within sheath 1044. As described above, the axial positions of outer needle 1046 and inner needle 1048 are controlled by the locations of outer needle shuttle 1066 and inner needle shuttle 1062. In this arrangement, outer needle shuttle 1066 and inner needle shuttle 1062 are positioned in their proximal-most locations.

In FIG. 12B, both outer needle shuttle 1066 and inner needle shuttle 1062 have translated distally from their prior positions shown in FIG. 12A. Therefore, both outer needle 1046 and inner needle 1048 have translated distally by the same amounts as outer needle shuttle 1066 and inner needle shuttle 1062. For example, it can be seen in the magnified view that outer needle 1046 (and inner needle 1048 encapsulated within outer needle 1046) are extended distally out from the confines of sheath 1044. The transition from the arrangement of FIG. 12A to the arrangement of FIG. 12B can be accomplished by the activation of both drive motors 1050 and 1052. Drive motors 1050 and 1052 can be activated simultaneously or sequentially. The transition from the arrangement of FIG. 12A to the arrangement of FIG. 12B may be performed, for example, to pierce the airway and to locate the tips of needles 1046 and 1048 near the target tissue.

In FIG. 12C, inner needle shuttle 1062 has translated distally from its prior position shown in FIG. 12B. Therefore, inner needle 1048 has translated distally by the same amount as inner needle shuttle 1062. However, outer needle shuttle 1066 has not moved from its prior position shown in FIG. 12B. Therefore, inner needle 1048 in FIG. 12C is extended distally out from the confines of outer needle 1046 (as shown in the magnified view). The transition from the arrangement of FIG. 12B to the arrangement of FIG. 12C can be accomplished by the activation of inner needle drive motor 1052. As inner needle drive motor 1052 is rotating, inner needle lead screw 1058 and inner needle 1048 also rotate correspondingly. Therefore, it should be understood that, during the transition from the arrangement of FIG. 12B to the arrangement of FIG. 12C, inner needle 1048 is both rotating and translating. In some cases, the pitch of inner needle lead screw 1058 substantially matches the pitch of the spiral portion of inner needle 1048. In some cases, a pitch of about 0.2 millimeters/revolution to about 1.0 millimeters/revolution is used. In some cases, a pitch of about 0.6 millimeters/revolution to about 1.4 millimeters/revolution is used. In some cases, a pitch of about 1.0 millimeters/revolution to about 2.0 millimeters/revolution is used. In some cases, a pitch of more than 2.0 millimeters/revolution is used. The transition from the arrangement of FIG. 12B to the arrangement of FIG. 12C may be performed, for example, to extend inner needle 1048 into the target tissue.

In FIG. 12D, outer needle shuttle 1066 has translated distally from its prior position shown in FIG. 12C. Therefore, outer needle 1046 has translated distally by the same amount as outer needle shuttle 1066. However, inner needle shuttle 1062 has not moved from its prior position shown in FIG. 12C. Therefore, outer needle 1046 in FIG. 12D is extended distally to re-encapsulate inner needle 1048 (as shown in the magnified view). The transition from the arrangement of FIG. 12C to the arrangement of FIG. 12D can be accomplished by the activation of outer needle drive motor 1050. The transition from the arrangement of FIG. 12C to the arrangement of FIG. 12D may be performed, for example, to shear tissue at the interface between the outer diameter of inner needle 1048 and the inner diameter of outer needle 1046. In addition, the transition from the arrangement of FIG. 12C to the arrangement of FIG. 12D may be performed to encapsulate and retain the sample tissue material contained in the interstitial areas between the spirals of inner needle 1048.

In FIG. 12E, both outer needle shuttle 1066 and inner needle shuttle 1062 have translated proximally from their prior positions shown in FIG. 12D. Therefore, both outer needle 1046 and inner needle 1048 have translated proximally by the same amounts as outer needle shuttle 1066 and inner needle shuttle 1062. For example, it can be seen in the magnified view that outer needle 1046 (and inner needle 1048 encapsulated within outer needle 1046) are re-encapsulated within the confines of sheath 1044. The transition from the arrangement of FIG. 12D to the arrangement of FIG. 12E can be accomplished by the activation of both drive motors 1050 and 1052. The transition from the arrangement of FIG. 12D to the arrangement of FIG. 12E may be performed, for example, to withdraw inner and outer needles 1048 and 1046 from the target tissue in preparation for the withdrawal of the entire needle biopsy system from the bronchoscope. In fact, with actuator 1000 in the arrangement shown in FIG. 12E, and the tissue sample material within the outer needle 1046, actuator 1000 can be decoupled from the bronchoscope handle and the needle assembly 1040 can be withdrawn from the instrument channel of the bronchoscope. Thereafter, the tissue sample material can be extracted by actuating drive motor 1052 to extend inner needle 1048 from the confines of outer needle 1046, thereby exposing the tissue sample material.

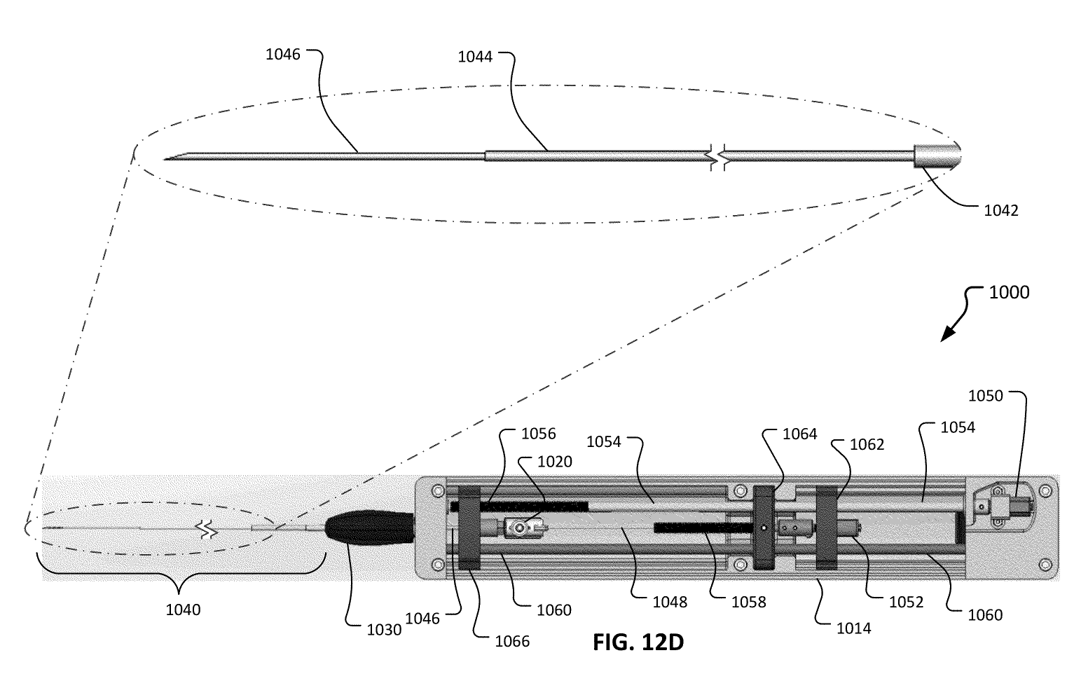

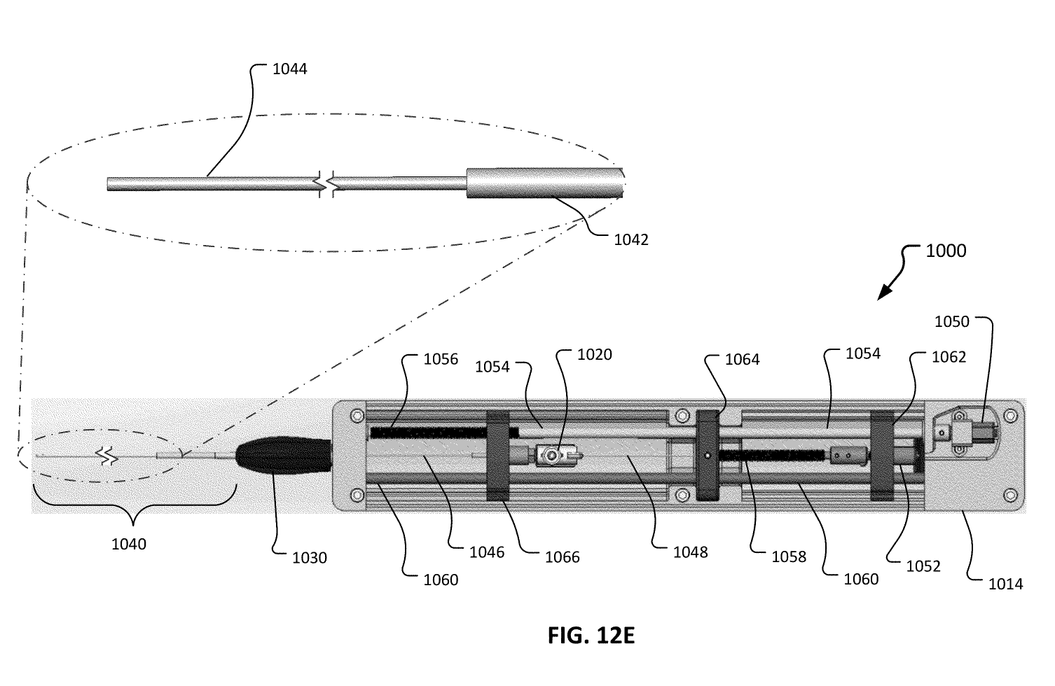

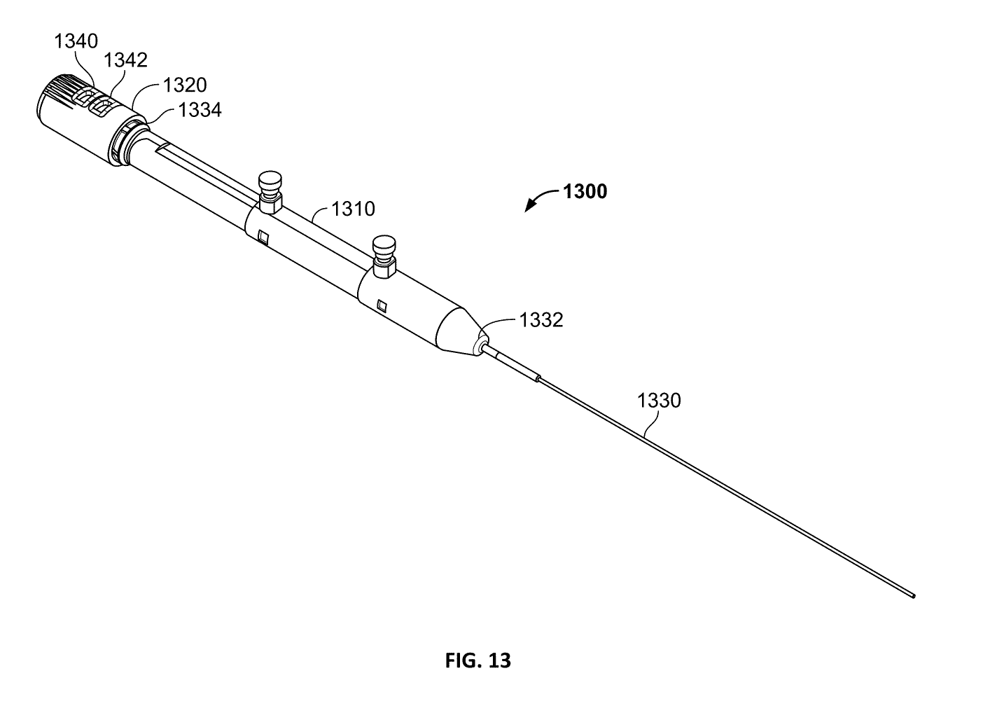

Referring to FIGS. 13-15, another exemplary actuator 1300 is provided that can be used with some cases of the needle biopsy systems provided herein. Actuator 1300 can include a housing 1310, a control knob 1320 and a needle assembly 1330. As shown, control knob 1320 is coupled to the proximal end portion 1332 of the housing. Needle assembly 1330 can extend from a distal end portion 1334 of housing 1310. As shown in FIG. 15, needle assembly 1330 can include an outer sheath 1344, an outer needle 1338, and an inner needle 1336.

Housing 1310 and control knob 1320 can be formed by the various methods and materials provided herein. Control knob 1320 can be directly coupled to the inner and outer needles 1336, 1338 of the needle assembly 1330. Control knob 1320 may be configured to advance and retract the inner and outer needles 1336, 1338 independently. In some implementations, control knob 1320 can be rotated, either clockwise or counterclockwise, to advance or retract inner needle 1336. In some implementations, control knob 1320 may be pushed forward to advance outer needle 1338 over inner needle 1336. Control knob 1320 optionally includes a release feature 1340, e.g., a release button, for proximally retracting outer needle 1338. Control knob 1320 optionally includes an indicator feature 1342 to provide a needle advancement distance or a length measurement to a user.

Still referring to FIG. 15, actuator 1300 is shown in a cross-sectional view to facilitate visualization of the internal components associated with motion actuation. Housing 1310 includes a main housing body 1346 and control knob 1320, in some cases. The components within main housing body 1346 can include, but are not limited to, outer needle 1338, inner needle 1336, a spring 1348, a threaded rod 1350, a needle drive 1352 and at least one drive rod 1354. These components are included in some exemplary actuator 1300 cases, but are not required in all actuator cases envisioned within the scope of this disclosure.

Outer needle 1338 can be coupled to needle drive 1352. Needle drive 1352 optionally slide proximally and distally within at least a portion of an interior of housing main body 1346. Movement of needle drive can be actuated by drive rod 1354, which can be coupled to control knob 1320. Accordingly, when control knob 1320 slides proximally, drive rod 1354 can advance needle drive 1352 and outer needle 1338 proximally.

Inner needle 1336 is optionally coupled to inner needle lead screw 1350. As inner needle lead screw 1350 rotates, inner needle 1336 can rotate in a corresponding fashion. Inner needle lead screw 1350 can be coupled to distal end portion 1334 of the main housing body 1346 in a threaded arrangement. That is, inner needle lead screw 1350 may have an external thread and main housing body 1354 that has a complementary internal thread. Accordingly, as inner needle lead screw 1350 rotates, inner needle lead screw 1350 and inner needle 1336 can translate proximally or distally in relation to housing 1310. It can be understood, therefore, that actuation of inner needle lead screw 1350 will cause inner needle 1336 to rotate and translate simultaneously.

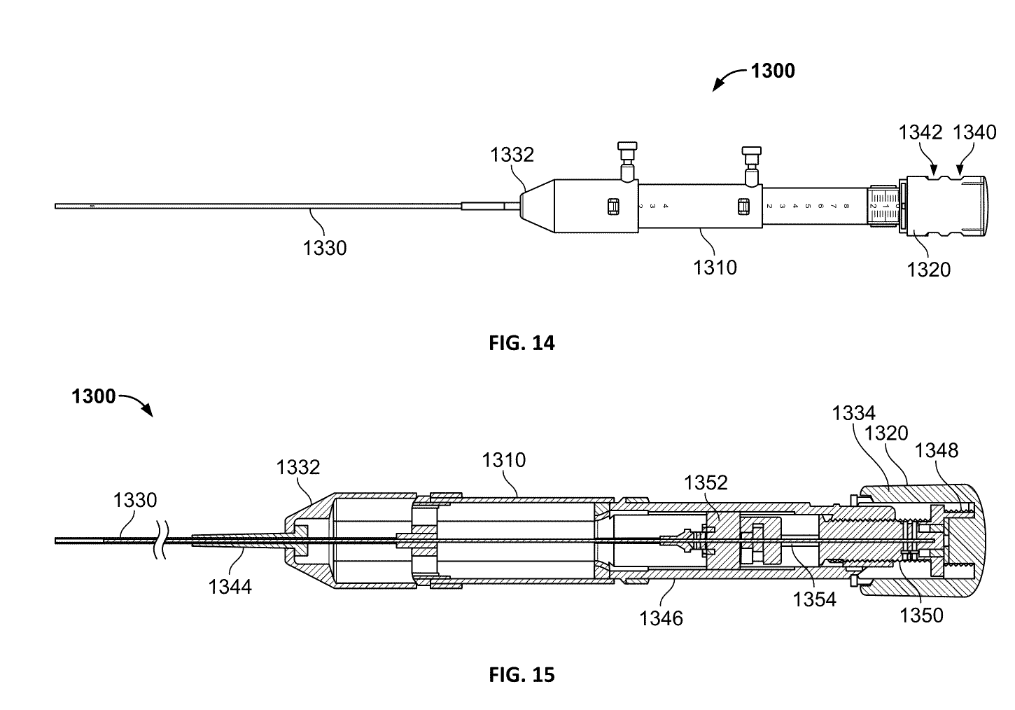

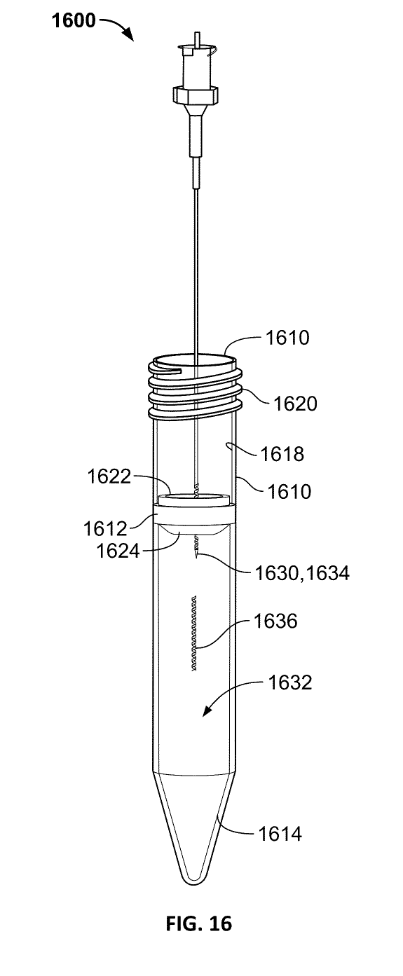

Referring to FIGS. 16-17, needle biopsy system cases provided herein may be used in conjunction with a tissue collection assembly 1600. Tissue collection assembly 1600 can include a vial 1610, a cap (not shown) and a septum 1620. Vial 1610 optionally includes a closed end 1614, an open end 1616 and an interior portion 1618. Interior portion 1618 of vial 1610 can have various suitable sizes and shapes. As shown in FIG. 16, interior portion 1618 of vial 1610 can be generally cylindrical with a conical profile at closed end 1614, in some cases. Open end 1616 of vial 1610 is optionally coupleable with cap. In some cases, vial 1610 includes a coupling feature 1620 at open end 1616 configured to mate with complementary features of cap. For example, coupling feature 1620 can be a threaded portion or a lip protrusion that mates with a complementary structure of cap, in some cases. Septum 1612 can comprise a flexible member with an outer surface 1622 and an inner surface 1624 and is configured to be disposed within interior portion 1618 of vial 1610. In various cases, at least a portion of septum 1612 can be sized and shaped complementary to interior portion 1618 of vial 1610 to facilitate sealing of open end 1616 of vial.

Still referring to FIGS. 16-17, distal tip 1630 of the needle biopsy system can be inserted into vial 1610 through open end 1616 and through septum 1612 into an interior cavity 1632 of tissue collection assembly 1600. In some cases, interior cavity 1632 is defined between closed end 1614 of vial 1610 and septum 1612 disposed within interior portion 1618 of vial 1610. Interior cavity 1632 can be configured to receive a biological sample. As such, needle biopsy system 1600 can deposit tissue sample material into interior cavity 1632. Septum 1612 is optionally configured to engage with inner needle 1634 by sealing around outer surface 1622 of the inner needle 1634. As the inner needle 1634 is withdrawn from interior cavity 1632, septum 1612 optionally engages with tissue sample material 1636 disposed on outer surface 1622 of inner needle 1634. When septum 1612 engages tissue sample material 1636, septum 1612 can allow inner needle 1630 to be withdrawn from interior cavity 1632 while preventing tissue sample material 1636 from passing through. Accordingly, tissue sample material 1636 can be retained within interior cavity 1632 of tissue collection assembly 1600 when inner needle 1630 is fully withdrawn from interior portion 1618 of vial 1610.

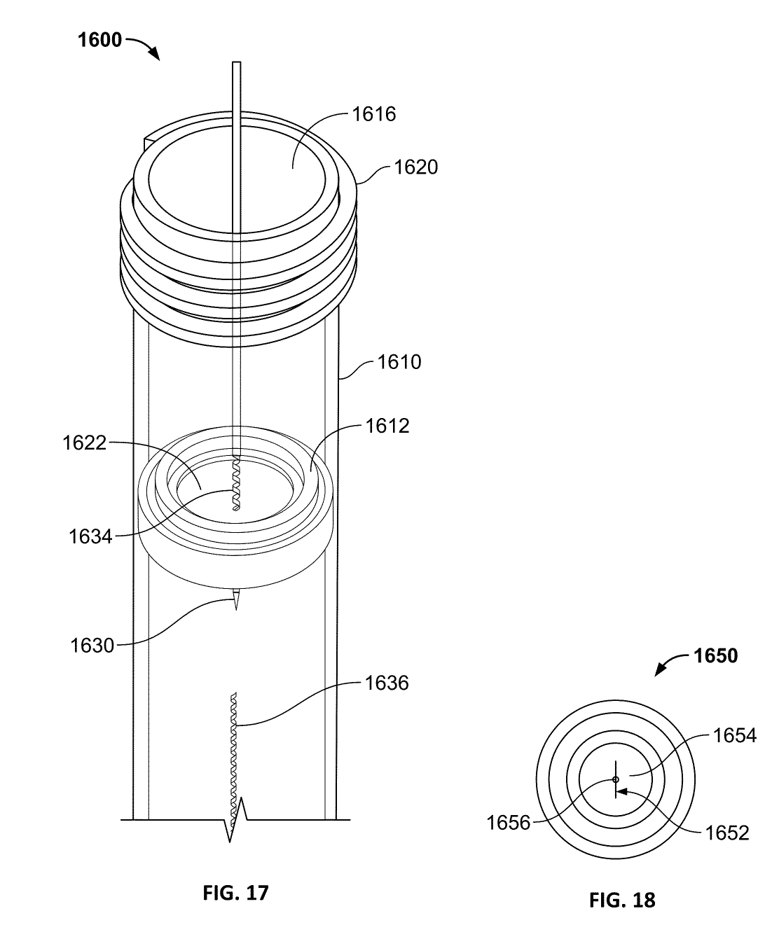

Referring to FIG. 18, an exemplary septum 1650 of a tissue collection assembly is provided that can be used with some cases of the tissue collection assembly provided herein. As shown, septum 1650 can comprise a slit formation 1652 for allowing a needle biopsy system to pass through septum 1650. In some cases, slit formation 1652 includes at least one slit 1654 that extends from an outer surface to an inner surface of septum 1650. Slit formation 1652 can allow portions of septum 1650 that are located proximate to slit 1654 to deflect outwardly or inwardly such that a distal end of a needle biopsy system can pass through septum 1650. In some cases, slit formation 1652 can comprise multiple slits that define a plurality of flaps configured to deflect outwardly or inwardly to create an opening for a needle biopsy system to pass through septum 1650. In some cases, septum 1650 can be made of a biocompatible, pliable polymer, for example, a silicone, polyurethane, polypropylene, polyethylene or a combination thereof.

In some cases, septum 1650 is optionally configured to reseal a needle puncture and therefore has no slit formation. For example, septum 1650 can reseal after a needle biopsy assembly passes through septum 1650 such that septum 1650 is able to retain any liquids or biological tissue sealed within an interior cavity of a tissue collection assembly. Septum 1650 may have a uniform or a variable thickness. For example, septum 1650 can have a thickness that increases in a radial direction relative to a central axis 1656. Decreasing the thickness of septum 1650 in a selected area, e.g., a central portion of septum 1650, can facilitate passing of a needle biopsy assembly through the selected area. Septum 1650 can generally provide a benefit of allowing a needle biopsy assembly to pass while sealing any liquids and tissue samples in an interior cavity of a tissue collection assembly.

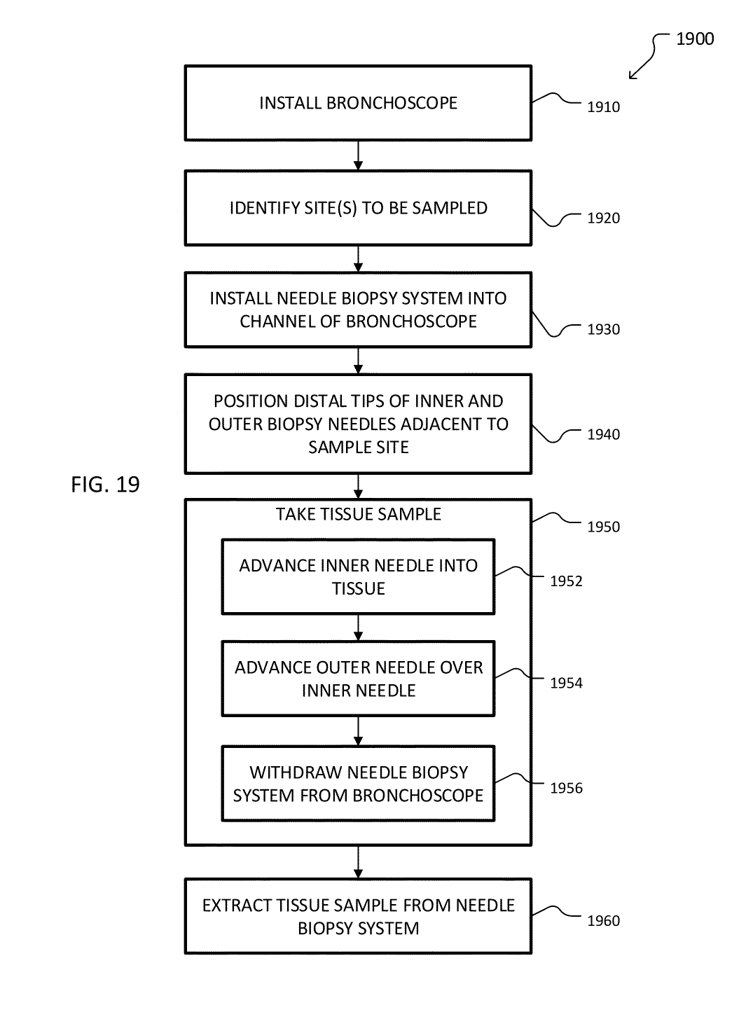

FIG. 19 is a flowchart of a method 1900 for performing a tissue biopsy procedure using a needle biopsy system in accordance with some cases provided herein. At operation 1910, a bronchoscope is installed in a patient. In some implementations, an EBUS bronchoscope is used. However, in other implementations an optical bronchoscope can be used. At operation 1920, the tissue site to be sampled is identified. For example, if an EBUS bronchoscope is being used, ultrasonic visualization can be used to identify the target tissue site. At operation 1930, a needle biopsy system as provided herein is installed into a channel of the bronchoscope. In result, for example, the arrangement of bronchoscope 30 and needle biopsy system 50 as illustrated in FIG. 1 can be attained. At operation 1940, the distal tips of the inner and outer biopsy needles are positioned adjacent to the target tissue area to be sampled. This can be performed, for example, by extending the inner and outer biopsy needles to the arrangement depicted in FIG. 12B.

Operation 1950 comprises taking a tissue sample. This can include a series of steps. First, at operation 1952, the inner needle is advanced into the tissue. This can be performed, for example, by extending the inner biopsy needle to the arrangement depicted in FIG. 12C. As the inner needle is extending distally, it is also rotating. This combination of translational and rotational motions results in movement by the inner needle that is similar to a screw-like motion. At operation 1954, the outer needle is advanced over the inner needle. This can be performed, for example, by extending the outer biopsy needle to the arrangement depicted in FIG. 12D. This operation may be performed to encapsulate and retain the sample tissue material contained in the interstitial areas between the spirals of the inner needle. In operation 1956, the needle biopsy system is withdrawn from the bronchoscope. In some cases, this operation include the retraction of the inner and outer needles into a sheath of the needle biopsy system as described in reference to FIG. 12E. The sample tissue material remains contained within the outer needle.

In operation 1960, the tissue sample is optionally extracted from the needle biopsy system. For example, the tissue sample material can be extracted by extending the inner needle from the confines of outer needle, thereby exposing the tissue sample material. The tissue extraction method will be discussed in greater detail in subsequent sections.

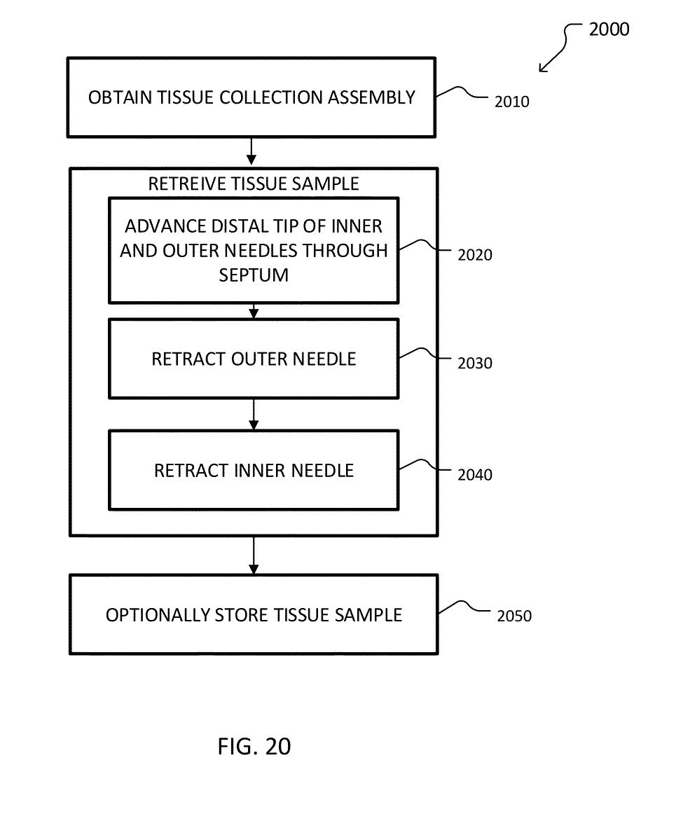

FIG. 20 is a flowchart of a method 2000 for extracting tissue sample material from a needle biopsy system in accordance with some cases provided herein. At operation 2010, a tissue collection assembly as provided herein is obtained. At operation 2020, distal tips of inner and outer biopsy needles of needle biopsy system are inserted into a vial of tissue collection assembly and through a septum of tissue collection assembly. At operation 2030, outer needle is retracted, using an actuator of needle biopsy system. Outer needle is retracted and removed from an interior cavity of tissue collection assembly. Retracting outer needle exposes a tissue sample material that is disposed over a outer surface of inner needle. At operation 2040, inner needle is retracted using actuator of needle biopsy system. As inner needle is retracted from interior cavity, tissue sample material engages with septum such that inner needle passes through the septum while tissue sample material remains within interior cavity of tissue collection assembly. At operation 2050, vial containing the tissue sample material can be capped and tissue collection assembly can be optionally stored for future testing.

While this specification contains many specific implementation details, these should not be construed as limitations on the scope of any invention or of what may be claimed, but rather as descriptions of features that may be specific to particular cases of particular inventions. Certain features that are described in this specification in the context of separate cases can also be implemented in combination in a single embodiment. Conversely, various features that are described in the context of a single embodiment can also be implemented in multiple cases separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the cases described herein should not be understood as requiring such separation in all cases, and it should be understood that the described program components and systems can generally be integrated together in a single product or packaged into multiple products.

Particular cases of the subject matter have been described. Other cases are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A needle biopsy system comprising:
an actuator device;
an outer needle with a lumen therethrough, wherein the outer needle extends distally from the actuator device; and
an inner needle at least partially disposed within the lumen, a distal tip of the inner needle capable of being fully disposed within the lumen, wherein the inner needle extends distally from the actuator device;
the actuator device being configured to translate the outer needle proximally and distally, and the actuator device being configured to translate the inner needle proximally and distally independently of the outer needle.

2. The needle biopsy system of claim 1, wherein the actuator device is configured to rotate the inner needle as the actuator device translates the inner needle.

3. The needle biopsy system of claim 1, wherein the inner needle includes a distal end portion with a spiral configuration.

4. The needle biopsy system of claim 1, wherein the inner needle includes a distal end portion with interstitial spaces that are configured to retain tissue material.

5. The needle biopsy system of claim 1, wherein the actuator includes an outer needle drive motor and an inner needle drive motor, wherein the outer needle drive motor and the inner needle drive motor are not the same motor.

6. The needle biopsy system of claim 5, wherein the actuator includes a power source that supplies electrical current to the outer needle drive motor and the inner needle drive motor.

7. The needle biopsy system of claim 1, wherein a spatial relationship between the inner needle and the outer needle is configured for shearing tissue therebetween.

8. The needle biopsy system of claim 1, wherein the outer needle comprises a tubular body having a plurality of slots formed therein, wherein the plurality of slots are configured to provide flexibility of the tubular body.

9. The needle biopsy system of claim 1, wherein a distal tip of the inner needle is positioned within the lumen of the outer needle in a first arrangement, and the distal tip of the inner needle is positioned out of the lumen of the outer needle in a second arrangement.

10. The needle biopsy system of claim 9, wherein the system is configured to move the outer and inner needles between a first and second arrangements by actuating the outer needle drive motor and the inner needle drive motor.

11. The needle biopsy system of claim 1, wherein the inner needle includes a distal end portion with a spiral configuration.

12. The needle biopsy system of claim 1, wherein the inner needle includes a distal end portion with interstitial spaces that are configured to retain tissue material.

13. The needle biopsy system of claim 1, wherein the inner needle has a main body portion and a tapered distal portion located proximal to the distal end portion of the inner needle, the tapered distal portion having a smaller diameter than a main body portion.

14. The needle biopsy system of claim 1, wherein a spatial relationship between the outer needle and the inner needle is configured for shearing tissue therebetween.

15. A method of collecting a tissue sample from a needle biopsy system comprising an actuator, an outer needle and an inner needle, the method comprising:
inserting the needle biopsy system into a tissue collection apparatus, the tissue collection apparatus comprising a vial, a flexible septum and an interior cavity configured to receive a biological sample, the interior cavity being defined by a closed end portion of the vial and the flexible septum disposed within an interior portion of the vial;
inserting the needle biopsy system through the septum into the interior cavity;
retracting the outer needle, using the actuator, from the interior cavity and exposing a tissue sample disposed over the outer surface of the inner needle;
retracting the inner needle, using the actuator, from the interior cavity and engaging the septum with the tissue sample such that the tissue sample remains within the interior cavity of the vial.

16. The method of claim 15, wherein the needle biopsy system is retracted through the septum that is self-sealing such after the needle biopsy passes through the septum, the septum is able to retain any liquids or biological tissue sealed within the interior cavity.

17. The method of claim 15, wherein the needle biopsy system is inserted or retracted through the septum that comprises a slit formation, the slit formation defining a plurality of flaps configured to deflect outwardly or inwardly to create an opening for the needle biopsy passing through the septum.

18. The method of claim 15, wherein the needle biopsy system is inserted through the septum that is sized and shaped complementary to the interior cavity of the vial.

19. A method of obtaining a tissue sample from a subject, the method comprising:
installing a bronchoscope into the subject;
identifying, using the bronchoscope, a tissue area from which to obtain the tissue sample;
installing a needle biopsy system into a channel of the bronchoscope, wherein the needle biopsy system includes an actuator, an outer needle, and an inner needle;
positioning a distal tip of the outer needle and a distal tip of the inner needle adjacent to the tissue area, wherein the inner needle is at least partially within a lumen of the outer needle;
advancing, using the actuator, the inner needle into the tissue area, wherein the inner needle simultaneously translates distally and rotates while advancing;
advancing, using the actuator, the outer needle into the tissue area such that the distal tip of the inner needle is within the lumen of the outer needle, wherein advancing the outer needle shears tissue between the inner and outer needles such that the tissue sample is disposed within the lumen of the outer needle; and
withdrawing the needle biopsy system from the bronchoscope.

20. The method of claim 19, further comprising extracting the tissue sample by advancing, using the actuator, the inner needle to remove the tissue sample from the lumen of the outer needle.
